(19) United States
(12) Patent Application Publication
(10) Pub. No.: US 2026/0007865 A1
(43) Pub. Date: Jan. 8, 2026

(54) SPIRAL GUIDEWIRES

(71) Applicant: SV SWISSVORTEX AG, Zürich (CH)

(72) Inventors: Andrea GUIDOTTI, Zurich (CH); Karl-Heinz KUCK, Hamburg (DE); Pietro GOZZOLI, Zürich (CH); Lucas FEUVRIER, Zürich (CH); Barbara ROSSER, Zürich (CH); Mattia ARDUINI, Zürich (CH); Luca VICENTINI, Opfikon (CH)

(73) Assignee: Schaffhauserstrasse 611, Zürich (CH)

(21) Appl. No.: 18/993,683

(22) PCT Filed: Jul. 13, 2023
(86) PCT No.: PCT/IB2023/057204

§ 371 Date: Jan. 13, 2025

Related U.S. Application Data
(60) Provisional application No. 63/388,714, filed on Jul. 13, 2022.

(51) Int. Cl. A61M 25/09; A61M 25/06

(52) U.S. Cl. CPC A61M 25/09; A61M 25/0662; A61M 2025/09058; A61M 2025/0915; A61M 2025/09175; A61M 2210/125

(57) ABSTRACT

A cardiac guidewire (20) is provided that is advanceable to a target cardiac site (10). The cardiac guidewire (20) is shaped so as to define an elongate portion (22), which is straight when unconstrained, and a spiral distal portion (24), which extends from and is supported by a distal end (26) of the elongate portion (22). The cardiac guidewire (20) is configured such that the spiral distal portion (24), when in an unconstrained resting state, is shaped as a spiral (30) that (a) spirals radially outward at an increasing distance from the distal end (26) of the elongate portion (22) so as to define an outer turn (32), and (b) has a zero or non-zero height. The cardiac guidewire (20) is further configured such that pushing of the elongate portion (22) in a distal direction (34), when the outer turn (32) of the spiral (30) engages cardiac tissue (36) at the target cardiac site (10), changes the height of the spiral (30) and deforms the spiral (30). Other embodiments are also described.

### CROSS-REFERENCE TO RELATED APPLICATIONS

The present application claims priority from U.S. Provisional Application 63/388,714, filed Jul. 13, 2022, which is assigned to the assignee of the present application and incorporated herein by reference.

### FIELD OF THE APPLICATION

The present invention relates generally to minimally-invasive surgical tools and techniques methods, and specifically to cardiac guidewires and methods of their use.

### BACKGROUND OF THE APPLICATION

Guidewires are widely used to facilitate the insertion of catheters into a vessel into the body. The guidewire is generally maneuvered proximally to facilitate its movement around or through the vessel path. A guidewire, or coil spring guide, is inserted into a vessel through a needle puncture site, until it reaches a certain target anatomic location, and a catheter is advanced over the guidewire until it reaches the target location Thereafter, the guidewire is either retracted from the body, leaving the catheter into the body, or the guidewire is kept in position during the catheter maneuvering. In other applications, the guidewire is inserted within a catheter already in place within a vessel, is then exposed until the guidewire reaches the target anatomy, the catheter is then retrieved, and the placed guidewire is used as guiding support for the advancement of a different catheter, typically a therapy catheter. The use of percutaneous entry not only for diagnostic purposed but also for therapeutic procedures has expanded rapidly, with the advancement of valvular treatments solutions or cardiovascular occlusion technologies. Generally guidewires are functional to control or influence the direction and advancement of these therapeutic catheters as they advance through the vessels, as supporting structure.

Large and rigid devices, such as transcatheter aortic valve replacement (TAVR) systems or left atrial appendage occluders (LAAo), are not influenced by soft guidewires. Stiff guidewires, which are stiffer than the vessels, are generally used with these types of therapeutic catheters, leading to possible vessels wall damage during their trajectory. In addition, the distal end of the guidewire can damage the target anatomy tissue against which the distal end is placed. During TAV, R the distal end of the guidewire is typically located against the base of the left ventricle: during LAAo the guidewire is typically nested within the left atrial appendage chamber. If the guidewire is misused, excessive pressure is applied to the guidewire, there is a fragile target anatomical wall tissue, or accidental damage is caused, the guidewire can cause direct trauma to the patient.

### SUMMARY OF THE APPLICATION

Some embodiments of the present invention provide a cardiac guidewire that is shaped so as to define a spiral distal portion, which when unconstrained is shaped as a spiral having a zero or non-zero height. The cardiac guidewire is intravascularly advanced toward a target cardiac site, such as a left atrial appendage (LAA) or a base of a ventricle, and positioned at the target cardiac site such that an outer turn of the spiral engages cardiac tissue at the target cardiac site. Further pushing of an elongate portion of the guidewire in a distal direction changes the height of the spiral and deforms the spiral while the outer turn of the spiral remains engaging the cardiac tissue.

By this change in height and deformation of the spiral, the spiral distal portion acts as a springy shock absorber, thereby inhibiting inadvertent excessive advancing of the guidewire that might perforate cardiac tissue at the target cardiac site, such as the LAA or the ventricular wall. For example, in the absence of the spiral distal portion, the distal tip of the guidewire might puncture, cut, or otherwise damage the cardiac tissue.

In addition, the engagement of the cardiac tissue by the outer turn of the spiral helps center the elongate portion of the guidewire within the LAA or the target cardiac chamber, thereby preventing inadvertent lateral cutting of the wall by the portion of the elongate portion within that chamber.

Still further, the engagement of the cardiac tissue by the outer turn of the spiral helps distribute the force of the guidewire against more tissue by increasing the contact surface between the guidewire and the tissue, typically around 360 degrees of the LAA. Typically, the surgeon advances the guidewire as far as possible within the LAA or cardiac chamber, until the guidewire comes in parietal contact with the tissue wall or the end of the chamber, in order to guarantee more stability of the guidewire during the further advancement of the catheter over the guidewire into the chamber, and not to lose the position of the guidewire, and thus of the target anatomy, during maneuvering of the catheter during its advancement.

The cardiac guidewire may be used for any cardiac procedure requiring a guidewire, such as placement of a cardiac implant, e.g., an LAA closure device, any cardiovascular occluder implant, or a prosthetic valve, e.g., a prosthetic aortic valve, mitral valve, tricuspid valve, pulmonic valve.

For some applications, the cardiac guidewire may also be used for applying energy, such as ablation energy, including but not limited to radiofrequency energy or cryogenic temperature energy, for ablating the target tissue wall anatomy, for cutting certain target anatomies, or for coagulating tissue. The guidewire can be placed within a pulmonary vein in the left atrium, or within a renal vein, completing at least one complete spiral revolution, to create a 360-degree contact with the vein tissue, and then used to apply an energy ablation of the tissue wall for the electrical isolation of the vein.

For some applications, the spiral distal portion, when unconstrained, is shaped as a planar spiral having the zero height or as a conical spiral having the non-zero height. In the latter case, when the spiral distal portion is unconstrained, an apex of the conical spiral may face proximally or distally.

There is therefore provided, in accordance with an application of the present invention, a cardiac guidewire, which is advanceable to a target cardiac site, and which is shaped so as to define:
an elongate portion, which is straight when unconstrained, and
a spiral distal portion, which extends from and is supported by a distal end of the elongate portion,
wherein the cardiac guidewire is configured such that:
   the spiral distal portion, when in an unconstrained resting state, is shaped as a spiral that (a) spirals radially outward at an increasing distance from the distal end of the elongate portion so as to define an outer turn, and (b) has a zero or non-zero height, and
   pushing of the elongate portion in a distal direction, when the outer turn of the spiral engages cardiac tissue at the target cardiac site, changes the height of the spiral and deforms the spiral.

For some applications, the cardiac guidewire is configured such that:
the spiral distal portion, when in the unconstrained resting state, is shaped as a planar spiral having the zero height,
the pushing of the elongate portion in the distal direction, when the outer turn of the spiral engages the cardiac tissue, increases the height of the spiral and deforms the spiral into a conical spiral having an apex that faces distally, and
pulling of the elongate portion in a proximal direction, when the outer turn of the spiral engages the cardiac tissue, increases the height of the spiral and deforms the spiral into a conical spiral in which the apex faces proximally.

For some applications, an interface portion of the cardiac guidewire between the elongate portion and the spiral distal portion has a radius of curvature of 5-30 mm when the spiral distal portion is in the unconstrained resting state.

For some applications, the spiral distal portion, when in the unconstrained resting state, has 1-6 turns.

For some applications, a pitch of the spiral distal portion, when in the unconstrained resting state, varies along a central longitudinal axis of the spiral distal portion.

For some applications, the elongate portion has a length of at least 70 cm.

For some applications, a distal end of the spiral distal portion, when in the unconstrained resting state, is shaped so as to define a hook.

For some applications, the cardiac guidewire is configured such that the spiral distal portion, when in the unconstrained resting state, is shaped as a conical spiral having the non-zero height.

For some applications, the cardiac guidewire is configured such that when the spiral distal portion is in the unconstrained resting state, the non-zero height of the spiral is at least 3 mm.

For some applications, the cardiac guidewire is configured such that when the spiral distal portion is in the unconstrained resting state, the non-zero height of the spiral equals at least 10% of a diameter of the outer turn of the spiral.

For some applications, the cardiac guidewire is configured such that when the spiral distal portion is in the unconstrained resting state, the spiral distal portion is shaped as a conical spiral having an apex that faces proximally, such that the pushing of the elongate portion in the distal direction, when the outer turn of the spiral engages the cardiac tissue at the target cardiac site, decreases the height of the conical spiral or inverts the conical spiral such that the apex faces distally.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the cardiac guidewire is configured such that when the spiral distal portion is in the unconstrained resting state, the spiral distal portion is shaped as a conical spiral having an apex that faces distally, such that the pushing of the elongate portion in the distal direction, when the outer turn of the spiral engages the cardiac tissue at the target cardiac site, increases the height of the conical spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the conical spiral is a hyperbolic conical spiral, which tapers hyperbolically from a base of the conical spiral to an apex of the conical spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the angle is 85-90 degrees.

For some applications, the elongate portion and the spiral distal portion have different stiffnesses.

For some applications, the elongate portion is stiffer than the spiral distal portion.

For some applications, the spiral distal portion is stiffer than the elongate portion.

For some applications, a radially-inner portion of the spiral distal portion and a radially-outer portion of the spiral distal portion have different stiffnesses.

For some applications, the radially-inner portion of the spiral distal portion is stiffer than the radially-outer portion of the spiral distal portion.

For some applications, a wire diameter of the spiral distal portion narrows toward a distal end of the spiral distal portion.

For some applications, the radially-outer portion of the spiral distal portion is stiffer than the radially-inner portion of the spiral distal portion.

For some applications, a wire diameter of the spiral distal portion widens toward a distal end of the spiral distal portion.

For some applications, a cardiac guidewire system is provided that includes the cardiac guidewire and further includes a delivery sheath in which the cardiac guidewire is removably disposed in a constrained delivery configuration, with the spiral distal portion held in an unwound elongate configuration by the delivery sheath.

There is further provided, in accordance with an application of the present invention, a method including:
intravascularly advancing a cardiac guidewire toward a target cardiac site, the cardiac guidewire shaped so as to define (a) an elongate portion, which is straight when unconstrained, and (b) a spiral distal portion, which extends from and is supported by a distal end of the elongate portion, wherein the cardiac guidewire is configured such that the spiral distal portion, when in an unconstrained resting state, is shaped as a spiral that (i) spirals radially outward at an increasing distance from the distal end of the elongate portion so as to define an outer turn, and (ii) has a zero or non-zero height; and
positioning the spiral distal portion at the target cardiac site such that the outer turn of the spiral engages cardiac tissue at the target cardiac site, such that pushing of the elongate portion in a distal direction changes the height of the spiral and deforms the spiral while the outer turn of the spiral remains engaging the cardiac tissue.

For some applications, the cardiac tissue is cardiac tissue of a left atrial appendage (LAA).

For some applications, the cardiac tissue is cardiac tissue of a base of a ventricle.

For some applications, the cardiac tissue is cardiac tissue of an ascending aorta.

For some applications, the cardiac tissue is cardiac tissue of a left ventricle for a TMVR (Transcatheter Mitral Valve Repair and Replacement) procedure.

For some applications, the cardiac tissue is cardiac tissue of a left ventricle for a TAVR (Transcatheter Aortic Valve Repair and Replacement) procedure.

For some applications, the cardiac tissue is cardiac tissue of a right ventricle for a TTVR (Transcatheter Tricuspid Valve Repair and Replacement) procedure.

For some applications:
the cardiac guidewire is configured such that the spiral distal portion, when in the unconstrained resting state, is shaped as a planar spiral having the zero height, and
positioning the spiral distal portion at the target cardiac site includes positioning the spiral distal portion at the target cardiac site such that the pushing of the elongate portion in the distal direction increases the height of the spiral and deforms the spiral into a conical spiral while the outer turn of the spiral remains engaging the cardiac tissue.

For some applications, the cardiac guidewire is configured such that the spiral distal portion, when in the unconstrained resting state, is shaped as a conical spiral having the non-zero height.

For some applications, the cardiac guidewire is configured such that when the spiral distal portion is in the unconstrained resting state, the non-zero height of the spiral is at least 3 mm.

For some applications, the cardiac guidewire is configured such that when the spiral distal portion is in the unconstrained resting state, the non-zero height of the spiral equals at least 10% of a diameter of the outer turn of the spiral.

For some applications, the cardiac guidewire is configured such that when the spiral distal portion is in the unconstrained resting state, the spiral distal portion is shaped as a conical spiral having an apex that faces proximally, such that the pushing of the elongate portion in the distal direction, when the outer turn of the spiral engages the cardiac tissue at the target cardiac site, decreases the height of the conical spiral or inverts the conical spiral such that the apex faces distally.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the cardiac guidewire is configured such that when the spiral distal portion is in the unconstrained resting state, the spiral distal portion is shaped as a conical spiral having an apex that faces distally, such that the pushing of the elongate portion in the distal direction, when the outer turn of the spiral engages the cardiac tissue at the target cardiac site, increases the height of the conical spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the conical spiral is a hyperbolic conical spiral, which tapers hyperbolically from a base of the conical spiral to an apex of the conical spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the angle is 85-90 degrees.

For some applications, an interface portion of the cardiac guidewire between the elongate portion and the spiral distal portion has a radius of curvature of 5-30 mm when the spiral distal portion is in the unconstrained resting state.

For some applications, the elongate and the spiral distal portion have different stiffnesses.

For some applications, the elongate portion is stiffer than the spiral distal portion.

For some applications, the spiral distal portion is stiffer than the elongate portion.

For some applications, a radially-inner portion of the spiral distal portion and a radially-outer portion of the spiral distal portion have different stiffnesses.

For some applications, the radially-inner portion of the spiral distal portion is stiffer than the radially-outer portion of the spiral distal portion.

For some applications, a wire diameter of the spiral distal portion narrows toward a distal end of the spiral distal portion.

For some applications, the radially-outer portion of the spiral distal portion is stiffer than the radially-inner portion of the spiral distal portion.

For some applications, a wire diameter of the spiral distal portion widens toward a distal end of the spiral distal portion.

For some applications, the spiral distal portion, when in the unconstrained resting state, has 1-6 turns.

For some applications, a pitch of the spiral distal portion, when in the unconstrained resting state, varies along a central longitudinal axis of the spiral distal portion.

For some applications, the elongate portion has a length of at least 70 cm.

For some applications, a distal end of the spiral distal portion, when in the unconstrained resting state, is shaped so as to define a hook.

For some applications, intravascularly advancing the cardiac guidewire toward the target cardiac site includes intravascularly advancing the cardiac guidewire toward the target cardiac site while the cardiac guidewire is removably disposed in a delivery sheath with the spiral distal portion held in an unwound elongate configuration by the delivery sheath.

For some applications, the method further includes advancing a cardiac device over the cardiac guidewire while the outer turn of the spiral engages the cardiac tissue.

For some applications, the method further includes using the cardiac guidewire to apply ablation energy to the cardiac tissue while the outer turn of the spiral engages the cardiac tissue.

There is still further provided, in accordance with an application of the present invention, a cardiac guidewire, which is advanceable to a target cardiac site, and which is shaped so as to define:
an elongate portion, which is straight when unconstrained, and
a spiral distal portion, which, at an outer end thereof, extends from and is supported by a distal end of the elongate portion,
wherein the cardiac guidewire is configured such that:
   the spiral distal portion, when in an unconstrained resting state, is shaped as a conical spiral that (a) spirals radially inward from the outer end of the spiral distal portion and (b) defines an outer turn,
   pushing of the elongate portion in a distal direction, before the outer turn of the spiral engages cardiac tissue at the target cardiac site, brings an apex and a distal portion of the spiral distal portion in contact with the cardiac tissue, and
   further pushing of the elongate portion in a distal direction, after the outer turn of the spiral engages cardiac tissue at the target cardiac site, brings the outer turn of the spiral in contact with the tissue, thereby distributing distally-directed force against the tissue around the outer turn of the spiral.

For some applications, the cardiac guidewire is configured such that when the spiral distal portion is in the unconstrained resting state, a distal end portion of the elongate portion falls in, is parallel to, or defines an angle of less than 90 degrees with a best-fit plane defined by the outer turn of the spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the cardiac guidewire is configured such that when the spiral distal portion is in the unconstrained resting state, the conical spiral has a height of at least 3 mm.

For some applications, the cardiac guidewire is configured such that when the spiral distal portion is in the unconstrained resting state, the conical spiral has a height that equals at least 10% of a diameter of the outer turn of the spiral.

There is additionally provided, in accordance with an application of the present invention, a method including:
intravascularly advancing a cardiac guidewire toward a target cardiac site, the cardiac guidewire shaped so as to define (a) an elongate portion, which is straight when unconstrained, and (b) a spiral distal portion, which, at an outer end thereof, extends from and is supported by a distal end of the elongate portion, wherein the cardiac guidewire is configured such that the spiral distal portion, when in an unconstrained resting state, is shaped as a conical spiral that (i) spirals radially inward from the outer end of the spiral distal portion and (ii) defines an outer turn;
pushing the elongate portion in a distal direction, before the outer turn of the spiral engages cardiac tissue at the target cardiac site, so as to bring an apex and a distal portion of the spiral distal portion in contact with the cardiac tissue; and
further pushing the elongate portion in a distal direction, after the outer turn of the spiral engages cardiac tissue at the target cardiac site, so as to bring the outer turn of the spiral in contact with the tissue, thereby distributing distally-directed force against the tissue around the outer turn of the spiral.

For some applications, the cardiac tissue is cardiac tissue of a left atrial appendage (LAA).

For some applications, the cardiac tissue is cardiac tissue of a left ventricle.

For some applications, the cardiac tissue is cardiac tissue of an ascending aorta.

For some applications, the cardiac tissue is cardiac tissue of a left ventricle for a TMVR (Transcatheter Mitral Valve repair and replacement) procedure.

For some applications, the cardiac tissue is cardiac tissue of a left ventricle for a TAVR (Transcatheter Aortic Valve repair and replacement) procedure.

For some applications, the cardiac tissue is cardiac tissue of a right ventricle for TTVR (Transcatheter Tricuspid Valve repair and replacement).

For some applications, the cardiac guidewire is configured such that when the spiral distal portion is in the unconstrained resting state, a distal end portion of the elongate portion falls in, is parallel to, or defines an angle of less than 90 degrees with a best-fit plane defined by the outer turn of the spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the cardiac guidewire is configured such that when the spiral distal portion is in the unconstrained resting state, the conical spiral has a height of at least 3 mm.

For some applications, the cardiac guidewire is configured such that when the spiral distal portion is in the unconstrained resting state, the conical spiral has a height that equals at least 10% of a diameter of the outer turn of the spiral.

There is yet additionally provided, in accordance with an application of the present invention, a holder for stabilizing an elongate element selected from the group consisting of: a guidewire, a catheter, a shaft, and a tubular medical instrument, the holder including:
a first soft element; and
a second soft element,
wherein the first and the second soft elements are configured to stabilize a spatial position of the elongate element by friction created by the first and the second soft elements when compressed against each other.

For some applications:
the first soft element is moveable and the second soft element has a fixed location, and
the holder further includes:
   a spring mechanism connected to the first moveable soft element; and
   a knob configured to:
      cause, upon movement of the knob, compression of the first moveable soft element by positioning the elongate element against the second fixed soft element, and
      cause, upon release of the knob, release of the second moveable soft element, allowing the spring mechanism to push the first moveable soft element against the second fixed soft element, thereby creating compression between the first and the second soft elements that stabilizes the elongate element by friction.

For some applications, the second fixed soft element is configured to rotate in one or more directions, allowing relative advancement or retrieval of the elongate element compressed between the first and the second soft elements due to stabilizing friction between the first and the second soft elements.

For some applications, the holder is configured to stabilize the elongate element when positioned between the first and the second soft elements such that the first and the second soft elements create friction on the stabilized elongated element.

For some applications, the holder further includes a integrated tape or clamping mechanism, which is configured to bond the holder to an operating table when the holder is positioned on the operating table.

For some applications, the holder is shaped so as to define an integrated a slot for storing the elongate element and fixing the position on the operating table when not used.

The present invention will be more fully understood from the following detailed description of embodiments thereof, taken together with the drawings, in which:

### BRIEF DESCRIPTION OF THE DRAWINGS

FIGS. 1A-B are schematic illustrations of a cardiac guidewire, shown in a constrained shape within a delivery sheath in FIG. 1A, and in an unconstrained resting state, with its distal portion outside of the delivery sheath, in FIG. 1B, in accordance with an application of the present invention;

FIGS. 2A-E are schematic illustrations of respective configurations of the cardiac guidewire of FIGS. 1A-B, in accordance with respective applications of the present invention;

FIGS. 3A-B, which are schematic illustrations of a portion of a method of deploying and applying force to the cardiac guidewire of FIG. 2B at a target cardiac site, in accordance with an application of the present invention;

FIG. 3C is a schematic illustration of a possible state of the cardiac guidewire of FIGS. 3A-B at the target cardiac site during the method of deployment, in accordance with an application of the present invention;

FIG. 3D is a schematic illustration of a method of advancing a cardiac device over the cardiac guidewire of FIGS. 3A-B at the target cardiac site, in accordance with an application of the present invention;

FIGS. 4A-B are schematic illustrations of the cardiac guidewire of FIGS. 1A-B and FIG. 3A, within the left cardiac ventricle, in accordance with respective applications of the present invention;

FIGS. 5 is a schematic illustration of the cardiac guidewire of FIGS. 1A-B, positioned within a pulmonary vein, in accordance with an application of the present invention:

FIGS. 6A-C are schematic illustrations of alternative configurations of the cardiac guidewire of FIGS. 1A-B and FIG. 2A, in accordance with respective applications of the present invention;

FIGS. 7A-B are schematic illustrations of alternative configurations of the cardiac guidewire of FIG. 2C, in accordance with respective applications of the present invention;

FIGS. 8A-C are schematic illustrations of alternative configurations of the cardiac guidewire of FIG. 1A-B, in accordance with respective applications of the present invention;

FIGS. 9A-C are schematic illustrations of alternative configurations of the cardiac guidewire of FIG. 2D-E, in accordance with respective applications of the present invention;

FIGS. 10A-C are schematic illustrations of alternative configurations of the cardiac guidewire of FIGS. 1A-B, in accordance with respective applications of the present invention;

FIGS. 11A-B are schematic illustrations of alternative configurations of the cardiac guidewire of FIGS. 1A-B, in accordance with respective applications of the present invention;

FIGS. 12 is a schematic illustration of alternative configurations of the cardiac guidewire of FIGS. 1A-B, in accordance with respective applications of the present invention;

FIGS. 13A-B are schematic illustrations of alternative configurations of the cardiac guidewire of FIGS. 1A-B and 2A, in accordance with respective applications of the present invention;

FIGS. 13C-D are schematic illustrations of alternative configurations of the cardiac guidewire of FIG. 2C, in accordance with respective applications of the present invention;

FIGS. 14A-B are schematic illustrations of alternative configurations of the cardiac guidewire of FIG. 2C, providing a larger surface of contact with the cardiac target tissue in larger cardiac chambers, in accordance with respective applications of the present invention;

FIGS. 15A-B are schematic illustrations of alternative configurations of the cardiac guidewire of FIG. 2C, in accordance with respective applications of the present invention;

FIGS. 16A-B are schematic illustrations of alternative configurations of the cardiac guidewire of FIGS. 1A-B and 2A, in accordance with respective applications of the present invention;

FIGS. 17A-C are schematic illustrations of alternative configurations of the cardiac guidewire of FIG. 2C, in accordance with respective applications of the present invention;

FIGS. 18A-C are schematic illustrations of alternative configurations of the cardiac guidewire of FIG. 3A, in accordance with respective applications of the present invention;

FIGS. 19A-C are schematic illustrations of alternative configurations of the cardiac guidewire of FIGS. 1A-B and 2A, in accordance with respective applications of the present invention;

FIGS. 20A-B are schematic illustrations of alternative configurations of the cardiac guidewire of FIGS. 1A-B and/or FIGS. 2A-D deployed in a base of the ventricle for cardiac resynchronization application and/or cardiac sensing, in accordance with respective applications of the present invention;

FIGS. 21A-B are schematic illustrations of alternative configurations of the cardiac guidewire of FIGS. 1A-B and/or FIGS. 2A-D, in accordance with respective applications of the present invention. The distal end of guidewire is covered/coated with a porous tissue, and can function as a filter or a blood clot collector. Optionally, the tissue covering may be treated with a drug, and the guidewire can be used to deliver localized and targeted treatment to the cardiac structure and/or wall;

FIGS. 22A-B are schematic illustrations of a guidewire holder, in accordance with an application of the present invention;

FIGS. 23A-B are schematic illustrations of the guidewire holder of FIGS. 22A-B, with a guidewire inserted therein, in accordance with an application of the present invention;

FIGS. 24A-B are further schematic illustrations of the guidewire holder of FIGS. 22A-B, with a guidewire inserted therein, in accordance with an application of the present invention; and

FIGS. 25A-B are additional schematic illustrations of the guidewire holder of FIGS. 22A-B, in accordance with an application of the present invention.

### DETAILED DESCRIPTION OF APPLICATIONS

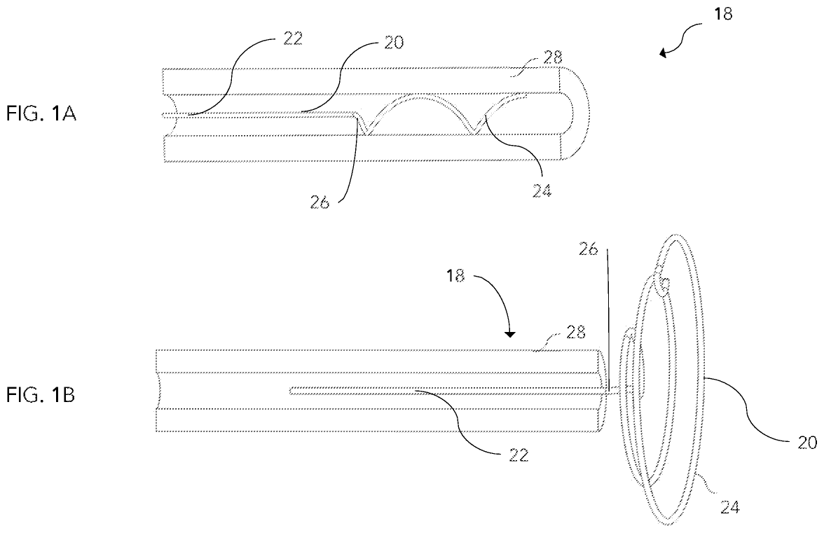

FIGS. 1A-B are schematic illustrations of a cardiac guidewire 20, in accordance with an application of the present invention. Cardiac guidewire 20 is pushable and advanceable to a target cardiac site, using transcatheter techniques known in the art. Cardiac guidewire 20 is shaped so as to define:
an elongate portion 22, which is straight when unconstrained, and
a spiral distal portion 24, which extends from and is supported by a distal end 26 of elongate portion 22.

FIG. 1A shows cardiac guidewire 20 removably disposed in a constrained delivery configuration within a delivery sheath 28 of a guidewire system 18, with spiral distal portion 24 held in an unwound elongate configuration by delivery sheath 28.

FIG. 1B shows cardiac guidewire 20 with spiral distal portion 24 in an unconstrained resting state upon deployment from a distal end of delivery sheath 28.

For some applications, guidewire 20 has a diameter of 0.014″-0.035″, such as 0.014″, 0.025″, or 0.035″.

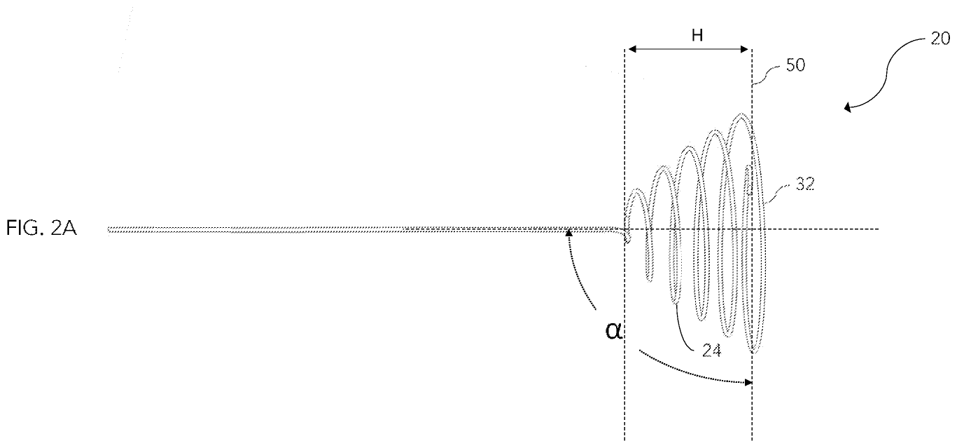

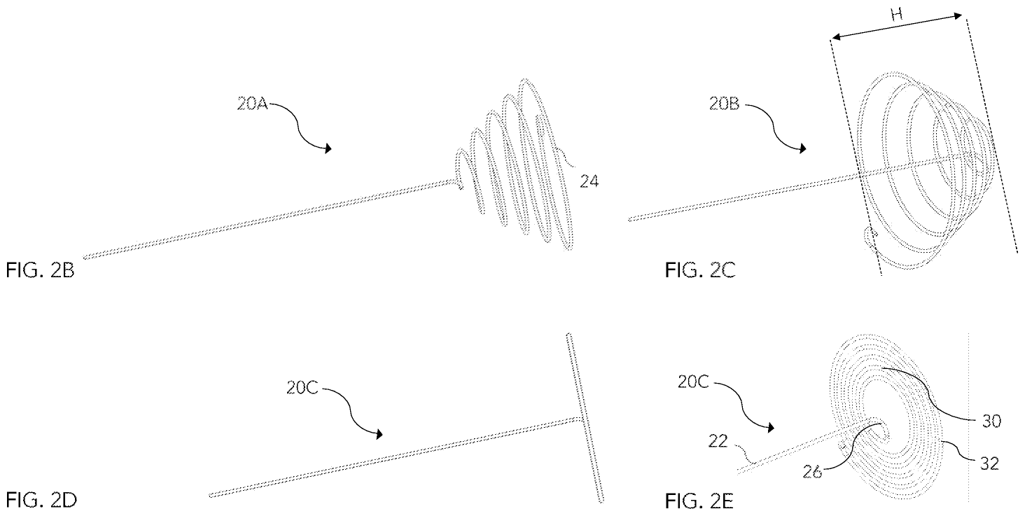

Reference is also made to FIGS. 2A, 2B, 2C, and 2D-E, which are schematic illustrations of cardiac guidewires 20, 20A, 20B, and 20C, respectively, in accordance with respective applications of the present invention. Cardiac guidewires 20A, 20B, and 20C are exemplary implementations of cardiac guidewire 20.

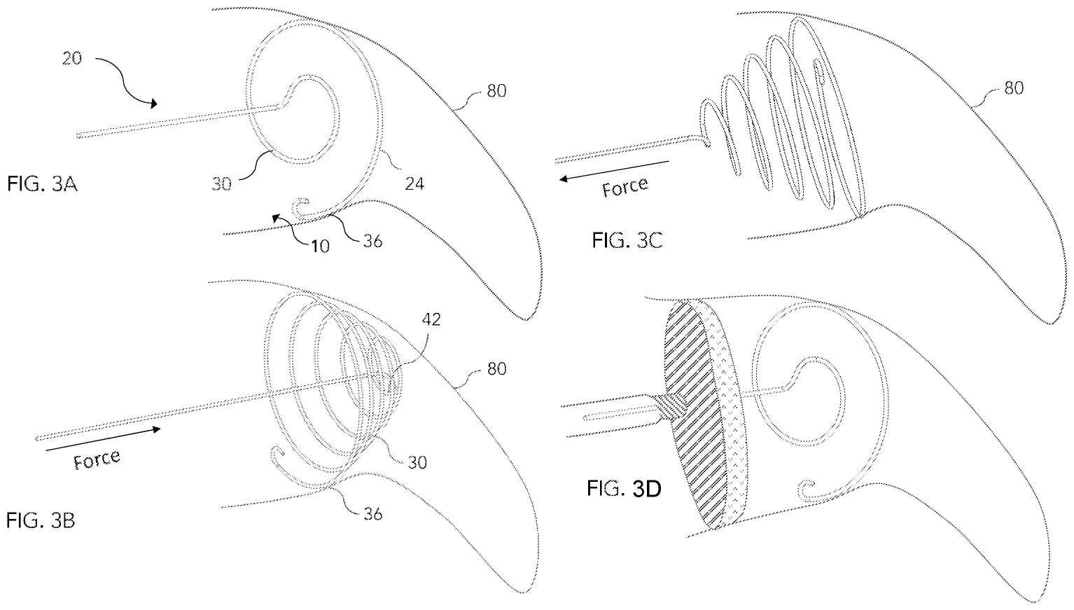

Reference is further made to FIGS. 3A-B, which are schematic illustrations of a portion of a method of deploying cardiac guidewire 20, 20A of FIG. 2B at a target cardiac site 10, in accordance with an application of the present invention. Reference is also made to FIG. 3C, which is a schematic illustration of a possible state of cardiac guidewire 20 at target cardiac site 10 during the method of deployment, in accordance with an application of the present invention. By way of example and not limitation, FIGS. 3A-C show cardiac guidewire 20C, shown in FIGS. 2D-E. Also by way of example and not limitation, target cardiac site 10 is shown as a left atrial appendage (LAA) 80.

Cardiac guidewire 20 is configured such that:
spiral distal portion 24, when in the unconstrained resting state, such as shown in FIG. 1B, is shaped as a spiral 30 that (a) spirals radially outward at an increasing distance from distal end 26 of elongate portion 22 so as to define an outer turn 32, and (b) has a zero heigh, such as shown in FIG. 2D-E, or non-zero height H, such as shown in FIGS. 2A-B, and
pushing of elongate portion 22 in a distal direction 34, when outer turn 32 of spiral 30 engages cardiac tissue 36 at target cardiac site 10, changes the height H of spiral 30 and deforms spiral 30, such as shown in FIG. 3B.

For some applications, spiral distal portion 24, when in the unconstrained resting state, has 1-6 turns.

For some applications, elongate portion 22 has a length of at least 70 cm.

For some applications, cardiac guidewire 20C is configured such that:
spiral distal portion 24, when in the unconstrained resting state, such as shown in FIGS. 2D-E and 3A, is shaped as a planar spiral 38 having the zero height,
pushing of elongate portion 22 in distal direction 34, when outer turn 32 of spiral 30 engages cardiac tissue 36, increases the height H of spiral 30 and deforms spiral 30 into a conical spiral 40 having an apex 42 that faces distally, such as shown in FIG. 3B, and
pulling of elongate portion 22 in a proximal direction 44, when outer turn 32 of spiral 30 engages cardiac tissue 36, increases the height H of spiral 30 and deforms spiral 30 into a conical spiral 46 in which apex 42 faces proximally, such as shown in FIG. 3C (this state may occur if outer turn 32 becomes engaged with target tissue in 36, thereby providing resistance during pulling).

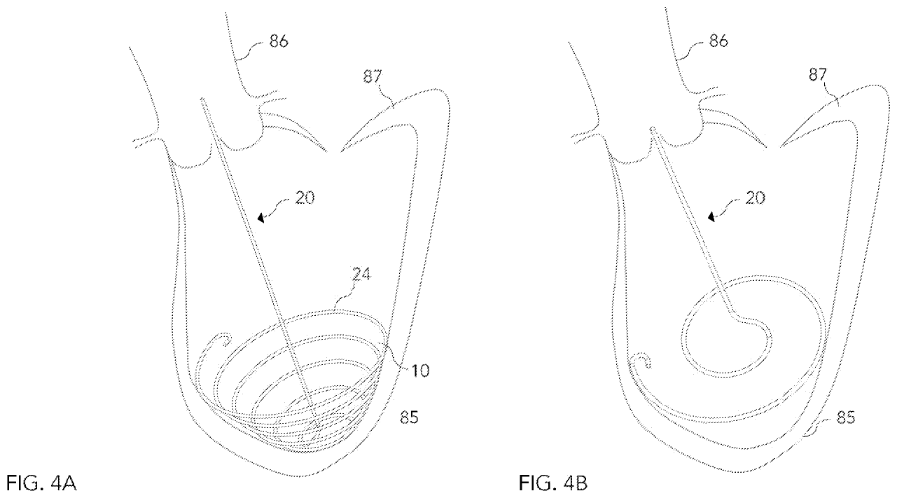

For some applications, each of conical spirals 40 and/or 42 is a linear conical spiral, in which the cone tapers linearly (constantly) from its base to apex 42, such as shown in FIGS. 1B, 2A, 3B, and 3C. For other applications, each of conical spirals 40 and/or 42 is a hyperbolic conical spiral, in which the cone tapers hyperbolically from its base to apex 42, such as described hereinbelow with reference to FIGS. 4A-B.

Reference is made to FIG. 2A. For some applications, cardiac guidewire 20A is configured such that spiral distal portion 24, when in the unconstrained resting state, is shaped as conical spiral 40 having the non-zero height H.

Reference is still made to FIG. 2A. For some applications, cardiac guidewire 20A is configured such that when spiral distal portion 24 is in the unconstrained resting state, the non-zero height H of spiral 30 is at least 3 mm and/or at least 10% of a diameter of outer turn 32 of spiral 30.

Reference is made to FIGS. 2A-C. For some applications, cardiac guidewire 20A, 20B is configured such that when spiral distal portion 24 is in the unconstrained resting state, a distal end portion 48 of elongate portion 22 defines an angle α (alpha) of 75-90degrees (e.g., 85-90 degrees, such as 90 degrees) with respect to a best-fit plane 50 defined by outer turn 32 of spiral 30 (labeled in FIG. 2A).

Reference is now made to FIG. 4A-B, which are schematic illustrations of guidewire 20 deployed in the left ventricle, in accordance with an application of the present invention. This deployment is typically used during TAVR procedures, but may also be used for other procedures.

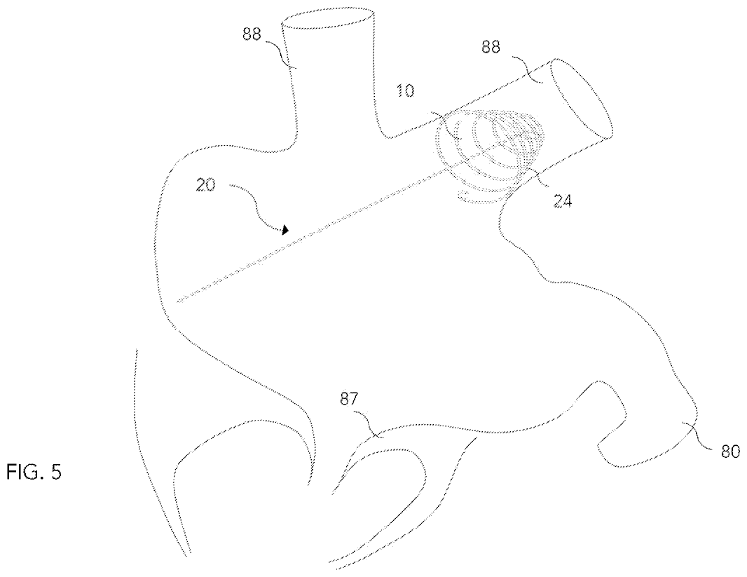

Reference is made to FIG. 5, which is a schematic illustration of guidewire 20 deployed in a pulmonary vein 88. When energy, e.g., radiofrequency (RF) energy or cryogenic energy, is applied to the guidewire, the guidewire creates a complete circumferential ablation lesion of the tissue, in order to treat rhythm disturbances.

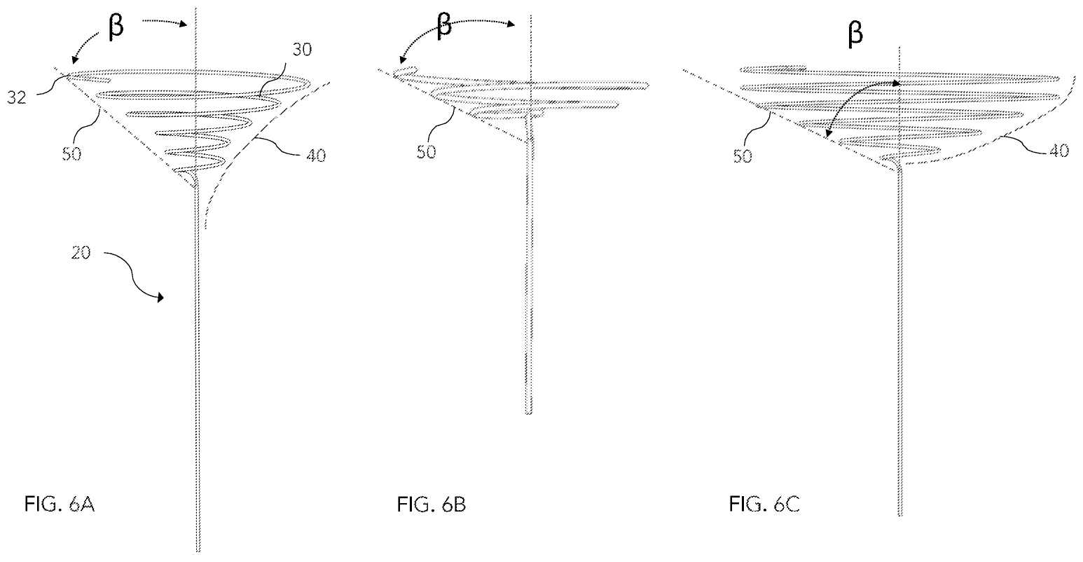

Reference is again made to FIG. 2A, and is additionally made to FIGS. 6A-C, which are schematic illustrations of alternative configurations of cardiac guidewire 20A, in accordance with respective applications of the present invention. For some applications, cardiac guidewire 20A is configured such that when spiral distal portion 24 is in the unconstrained resting state, spiral distal portion 24 is shaped as conical spiral 40 having apex 42 that faces proximally, such that the pushing of elongate portion 22 in distal direction 34, when outer turn 32 of spiral 30 engages cardiac tissue 36 at target cardiac site 10, decreases the height H of conical spiral 40 or inverts conical spiral 40 such that apex 42 faces distally.

For some applications, such as shown in FIG. 6A-C, conical spiral 40 is a hyperbolic conical spiral, with angle β (beta) between 0 and 90 degrees, as defined between the best-fit plane 50 defined by outer turn 32 of spiral 30, which tapers hyperbolically from a base 54 of conical spiral 40 to apex 42. The hyperbolic taper may be concave as viewed from outside, such as shown in FIG. 6A, or convex as viewed from outside, such as shown in FIG. 6C.

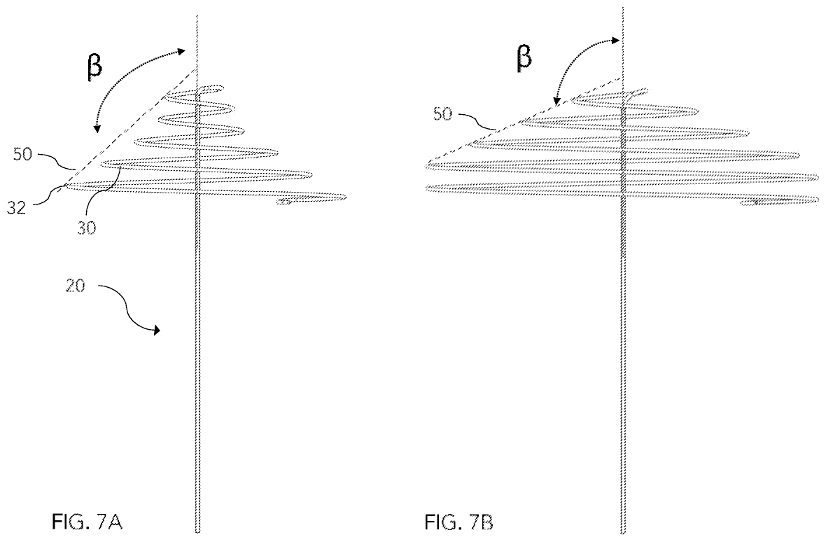

Reference is again made to FIG. 2C, and is additionally made to FIGS. 7A-B, which are schematic illustrations of alternative configurations of cardiac guidewire 20B, in accordance with respective applications of the present invention. For some applications, cardiac guidewire 20B is configured such that when spiral distal portion 24 is in the unconstrained resting state, spiral distal portion 24 is shaped as conical spiral 40 having apex 42 that faces distally, such that the pushing of elongate portion 22 in distal direction 34, when outer turn 32 of spiral 30 engages cardiac tissue 36 at target cardiac site 10, increases the height H of conical spiral 40.

Reference is made to FIGS. 2C, 5, 7A-B, 13D, 14A-B. In these configurations of guidewire 20, elongate portion 22 is connected to apex 42, such that the spiral around apex 42 acts as a spring for absorption of the pressure applied to elongate portion 22 of guidewire 20. In some particular configurations, the base of the spiral is configured to engage the tissue, and retain and/or create friction with the tissue, so as to reduce the pressure applied to the guidewire, thereby avoiding excessive pressure or movement of apex 42, thereby reducing the risk that the apex might perforate the tissue wall. Nevertheless, apex 42 can still reach a more distal and narrower target area, but, upon reaching this distal area, is constrained by be over-advanced into the distal wall of the cardiac structure, by contact between the proximal base of the spiral with surrounding cardiac tissue.

For some applications, such as shown in FIG. 5A-B, conical spiral 40 is a hyperbolic conical spiral, with angle β (beta) between 90 and 180 degrees, as defined between the best-fit plane 50 defined by outer turn 32 of spiral 30, in which the cone tapers hyperbolically from base 54 to apex 42. The hyperbolic taper may be concave as viewed from outside, such as shown in FIG. 7A, or convex as viewed from outside, such as shown in FIG. 7B.

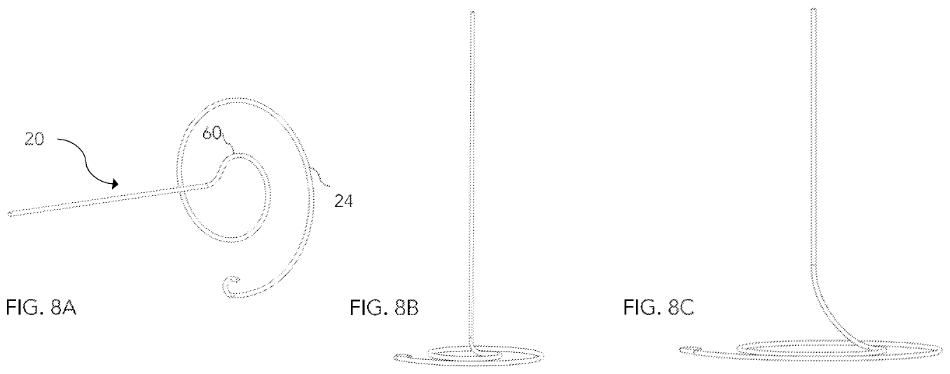

Reference is made to FIGS. 8A-C, which are schematic illustrations of alternative configurations of cardiac guidewire 20C, in accordance with respective applications of the present invention. Although the features of these configurations are illustrated for cardiac guidewire 20C, the features are equally applicable to cardiac guidewires 20A and 20B. In these configurations, an interface portion 60 of cardiac guidewire 20 between elongate portion 22 and spiral distal portion 24 has a radius of curvature of 5-30 mm when spiral distal portion 24 is in the unconstrained resting state, such as shown in FIGS. 6A-C. Each of FIGS. 8A-C shows a different exemplary radius of curvature of 5-30 mm. This radius of curvature may aid with recapture of spiral distal portion 24 (i.e., proximal withdrawal of spiral distal portion 24 into delivery sheath 28), which might be difficult under certain circumstances if interface portion 60 instead had a right angle.

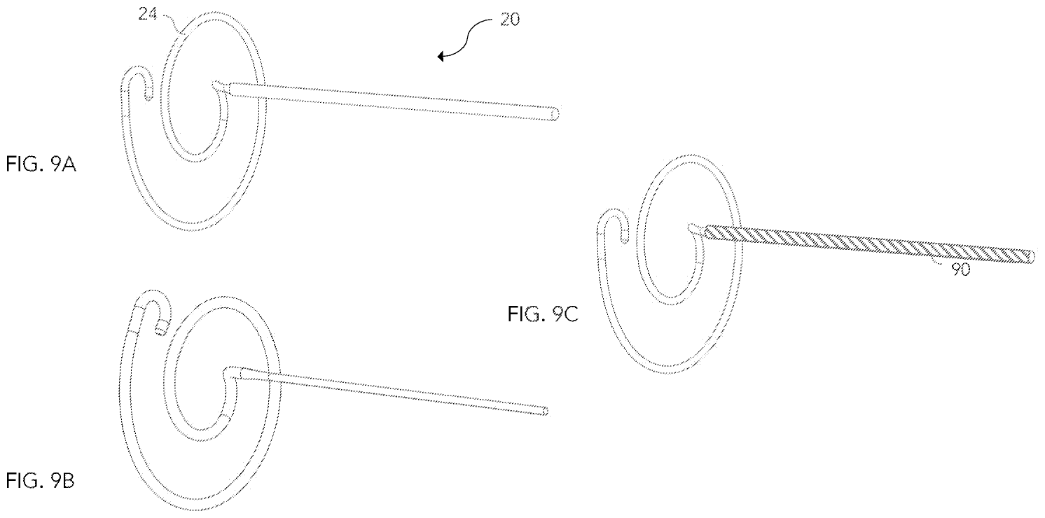

Reference is made to FIGS. 9A-C, which are schematic illustrations of alternative configurations of cardiac guidewire 20C, in accordance with respective applications of the present invention. Although the features of these configurations are illustrated for cardiac guidewire 20C, the features are equally applicable to cardiac guidewires 20A and 20B. In these configurations, elongate portion 22 and spiral distal portion 24 have different stiffnesses. For example, the different stiffnesses may be provided by different wire diameters (i.e., cross-sectional diameters), such as shown, and/or by different material properties or treatments, e.g., coating 90 as shown in FIG. 9C.

In the configuration shown in FIG. 9A, elongate portion 22 is stiffer than spiral distal portion 24.

In the configuration shown in FIG. 9B, wherein spiral distal portion 24 is stiffer than elongate portion 22.

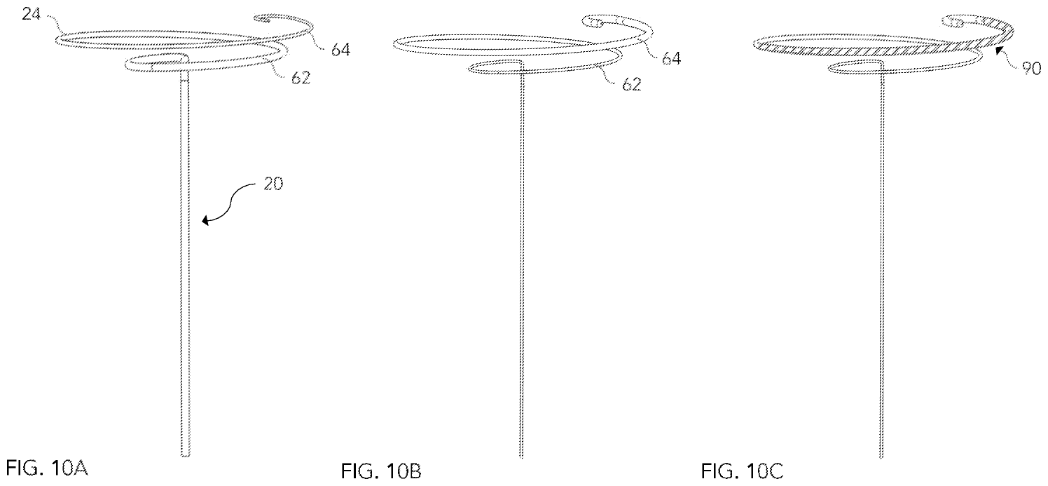

Reference is made to FIGS. 10A-C, which are schematic illustrations of alternative configurations of cardiac guidewire 20A, in accordance with respective applications of the present invention. Although the features of these configurations are illustrated for cardiac guidewire 20A, the features are equally applicable to cardiac guidewires 20B and 20C. In these configurations, a radially-inner portion 62 of spiral distal portion 24 and a radially-outer portion 64 of spiral distal portion 24 have different stiffnesses. For example, the different stiffnesses may be provided by different wire diameters, such as shown, and/or by different material properties or treatments, e.g., coating 90 as shown in FIG. 10C

In the configuration shown in FIG. 10A, radially-inner portion 62 of spiral distal portion 24 is stiffer than radially-outer portion 64 of spiral distal portion 24. For example, the wire diameter of spiral distal portion 24 may narrow toward a distal end 66 of spiral distal portion 24.

In the configuration shown in FIG. 10B, spiral distal portion 24 is stiffer than elongate portion 22. For example, the wire diameter of spiral distal portion 24 may widen toward distal end 66 of spiral distal portion 24.

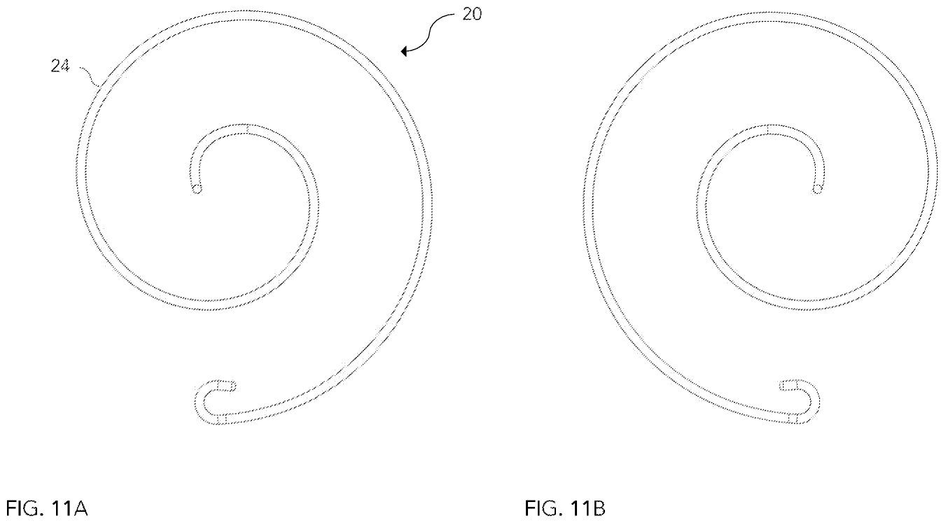

Reference is now made to FIGS. 11A-B, which are schematic illustrations of configurations of cardiac guidewire 20, in accordance with respective applications of the present invention. Spiral distal portion 24, when in the unconstrained resting state, may spiral in a clockwise or counterclockwise direction. All of the configurations of the guidewires described herein may spiral in a clockwise or counterclockwise direction. The 3D structure may improve manufacturability, use (to have clear rotation direction for deploying or retrieving), or for using its shape to navigate through the cardiac anatomy, i.e., rotating it when at the level of the aortic valve, can be used to easily cross the valve, since the spiral end will tend to enter into one commissure, then making its way to the left ventricle.

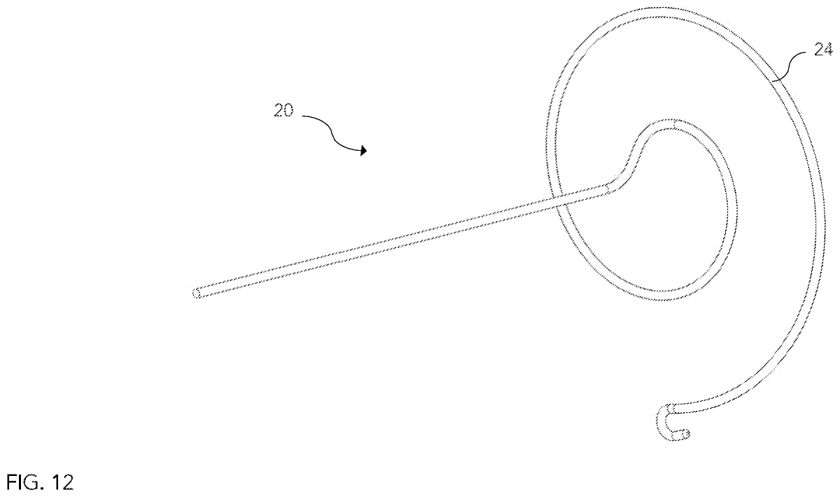

Reference is still made to FIGS. 11A-B, and additionally made to FIG. 12, which is a schematic illustration of another configuration of cardiac guidewire 20, in accordance with an application of the present invention. In the configurations shown in FIGS. 11A-B and 12, distal end 66 of spiral distal portion 24, when in the unconstrained resting state, is shaped so as to define a hook 70. In the configurations shown in FIGS. 9A-B, hook 70 curves radially inward, which may. In the configuration shown in FIG. 10, hook 70 curves radially outward, which may facilitate engagement with cardiac tissue 36 without causing damage to the tissue.

Typically, a radius of curvature of hook 70 is less than 40 mm.

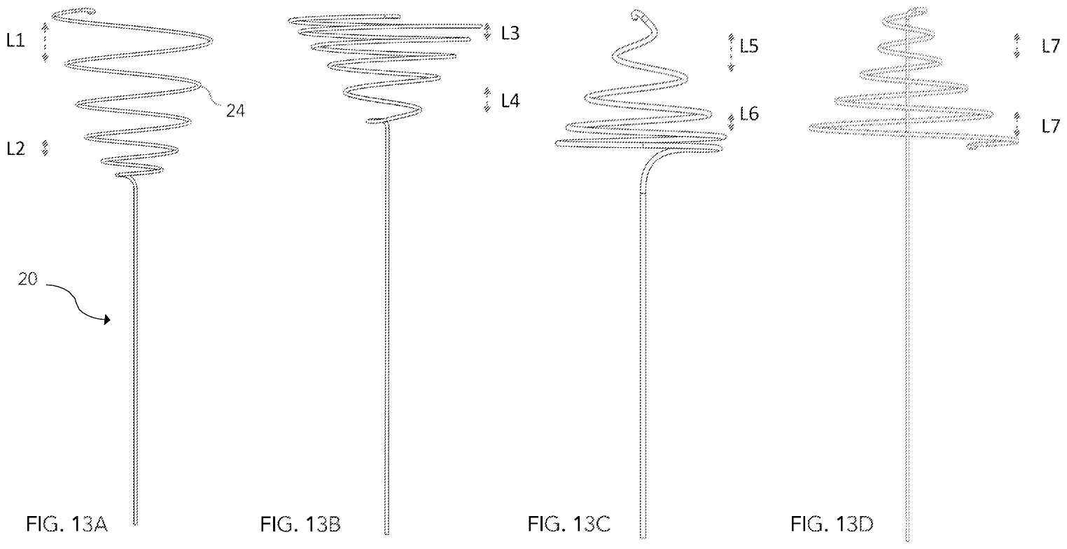

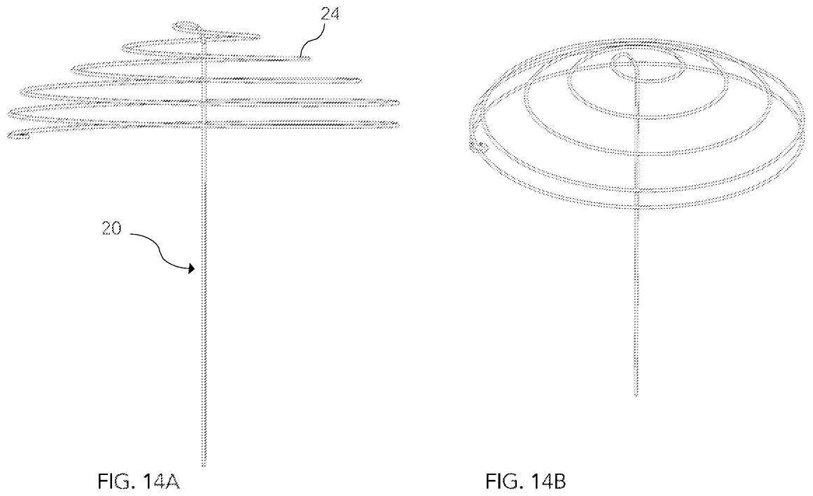

Reference is made to FIGS. 13A-D, which are schematic illustrations of alternative configurations of cardiac guidewire 20A (FIGS. 13A-B) and 20B (FIG. 13D), in accordance with respective applications of the present invention. In these configurations, a pitch of spiral distal portion 24, when in the unconstrained resting state, varies along a central longitudinal axis 74 (L1 vs. L2 in FIG. 13A, L3 vs. L4 in FIG. 13B, and L5 vs. L6 in FIG. 13C) (unlike the pitches shown in the other figures, which are constant along the central longitudinal axis). In FIG. 13D the pitch of the spiral is stable and not varying along the longitudinal axis (L7).

In the configuration shown in FIG. 13A, the pitch increases in distal direction toward distal end 66.

In the configuration shown in FIG. 13B, the pitch decreases in distal direction toward distal end 66.

Alternatively, the pitch may both decrease and increase in distal direction 34 along different portions of central longitudinal axis 74 (configuration not shown).

Reference is again made to FIGS. 3A-C. In an application of the present invention, a method is provided that comprises:
intravascularly advancing cardiac guidewire 20 toward target cardiac site 10; and
positioning spiral distal portion 24 at target cardiac site 10 such that outer turn 32 of spiral 30 engages cardiac tissue 36 at target cardiac site 10, such as shown in FIG. 3A, such that pushing of elongate portion 22 in a distal direction changes the height H of and deforms spiral 30 while outer turn 32 of spiral 30 remains engaging cardiac tissue 36, such as shown in FIG. 3B.

Typically, at least a portion of the advancing cardiac guidewire 20 comprises pushing cardiac guidewire 20 toward target cardiac site 10.

For some applications, cardiac guidewire 20 is configured such that spiral distal portion 24, when in the unconstrained resting state, is shaped as a planar spiral having the zero height H, and positioning spiral distal portion 24 at target cardiac site 10 comprises positioning spiral distal portion 24 at target cardiac site 10 such that pushing of elongate portion 22 in distal direction 34 increases the height H of spiral 30 and deforms spiral 30 into conical spiral 40 while outer turn 32 of spiral 30 remains engaging cardiac tissue 36, such as shown in FIG. 3B.

For some applications, cardiac guidewire 20 is intravascularly advanced toward target cardiac site 10 while cardiac guidewire 20 is removably disposed in delivery sheath 28 with spiral distal portion 24 held in an unwound elongate configuration by delivery sheath 28, such as shown in FIG. 1A.

Reference is now made to FIG. 3D. For some applications, the method further comprises advancing a cardiac device over cardiac guidewire 20 while outer turn 32 of spiral 30 engages cardiac tissue 36. For example, the cardiac device may be an LAA closure device 95, as shown in FIG. 3D, or a prosthetic cardiac valve.

For some applications, the method further comprises using cardiac guidewire 20 to apply ablation energy to cardiac tissue 36 while outer turn 32 of spiral 30 engages cardiac tissue 36, as described hereinabove with reference to FIG. 5.

For some applications, cardiac tissue 36 is cardiac tissue of an ascending aorta, such as during performance of endovascular aneurysm repair (EVAR), such as thoracic endovascular aortic aneurysm repair (TEVAR).

For some applications, cardiac tissue 36 is cardiac tissue of a left ventricle for a TMVR (Transcatheter Mitral Valve Repair and Replacement) procedure.

For some applications, cardiac tissue 36 is cardiac tissue of a left ventricle for a TAVR (Transcatheter Aortic Valve Repair and Replacement) procedure, such as shown in FIG. 4A-B.

For some applications, cardiac tissue 36 is cardiac tissue of a right ventricle for a TTVR (Transcatheter Tricuspid Valve Repair and Replacement) procedure.

For some applications, cardiac tissue 36 is cardiac tissue of an inferior vena cava (IVC) or superior vena cava (SVC) for a cava filter or a venous implant procedure.

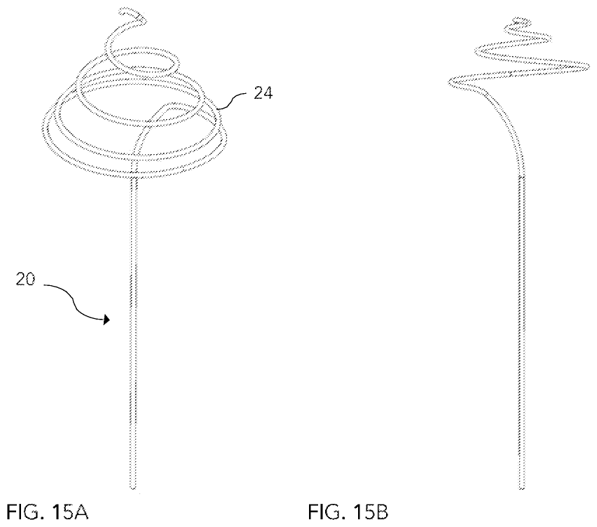

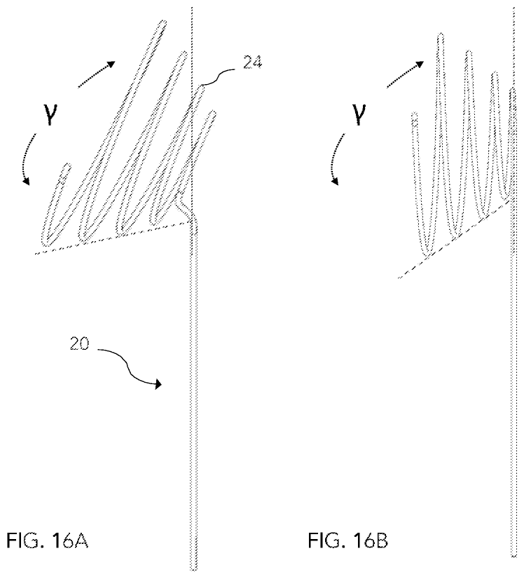

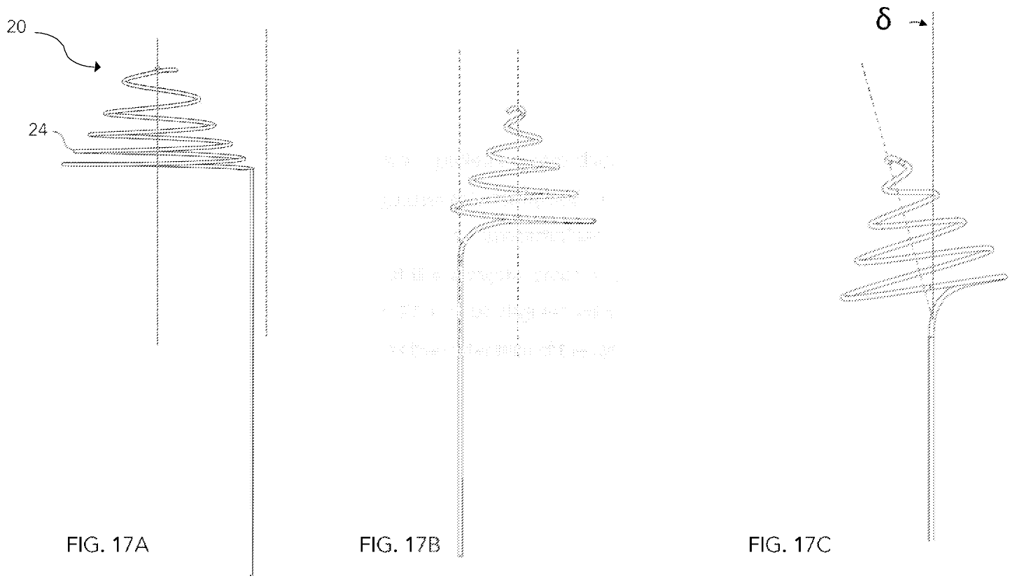

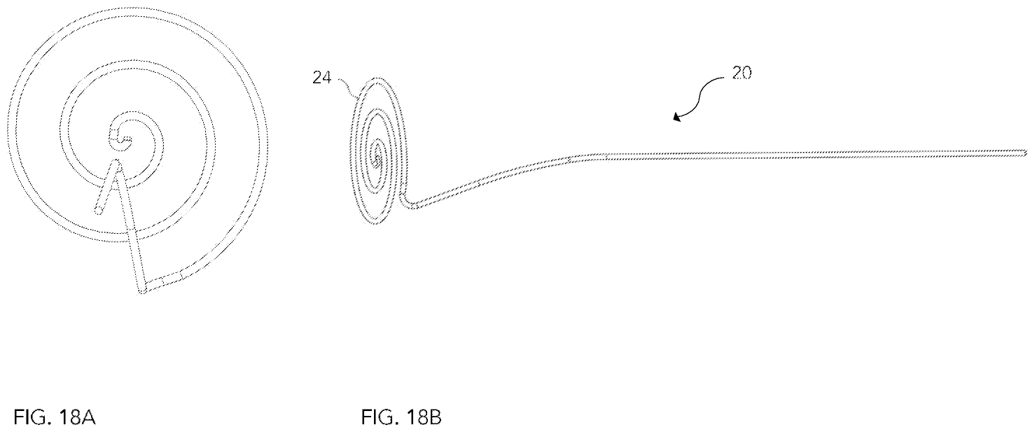

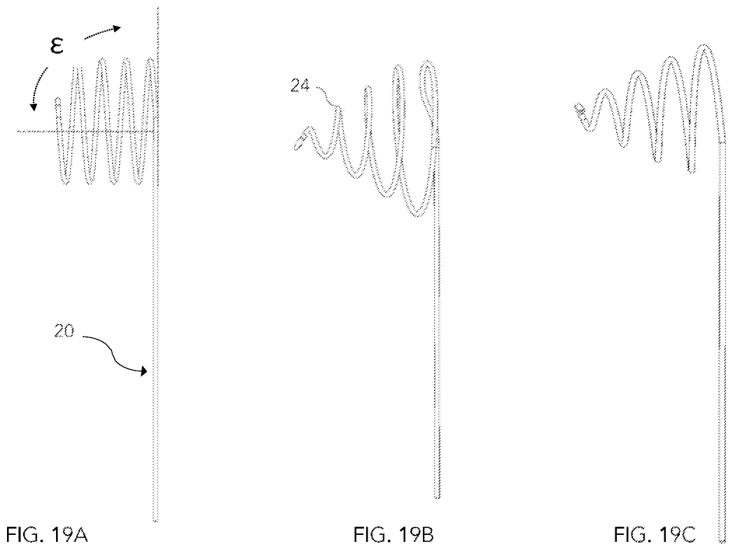

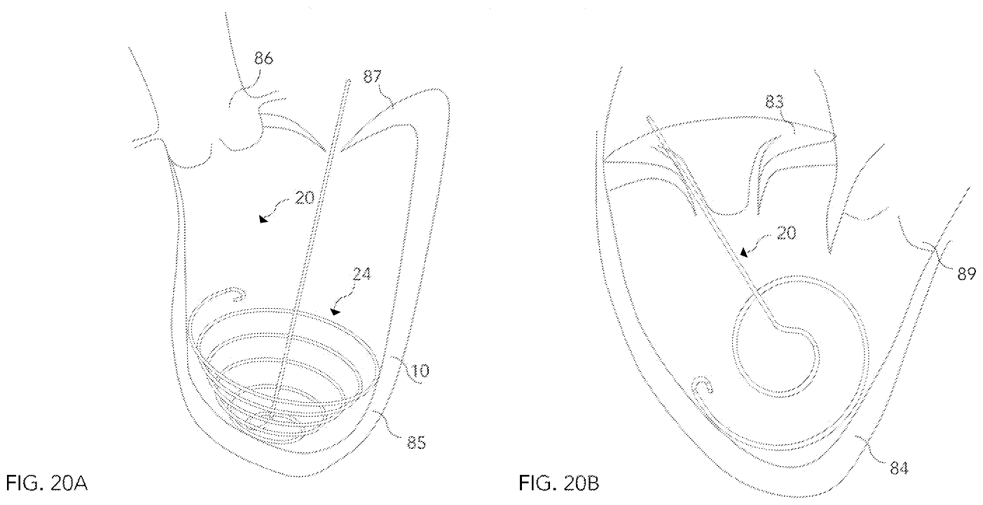

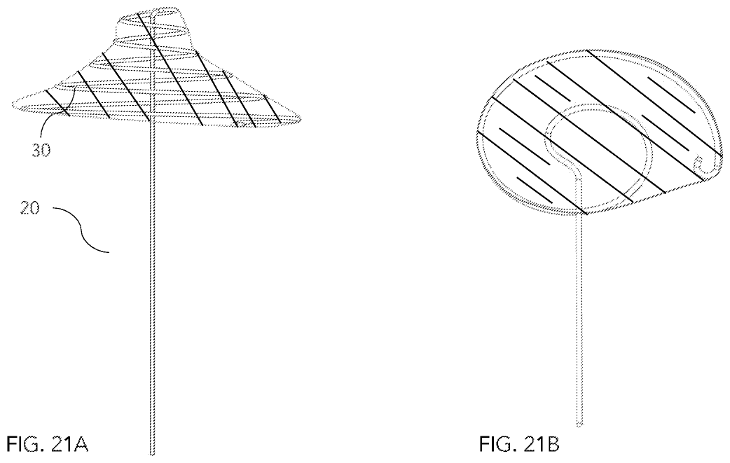

Reference is now made to FIGS. 13C, 15A-B, 17A-C, 18A-B, and 19A-C, which are schematic illustrations of alternative configurations of cardiac guidewire 20, in accordance with respective applications of the present invention.

In these configurations, cardiac guidewire 20, which is advanceable to a target cardiac site, is shaped so as to define an elongate portion, which is straight when unconstrained, and a spiral distal portion, which, at an outer end thereof, extends from and is supported by a distal end of the elongate portion. Cardiac guidewire 20 is configured such that:
the spiral distal portion, when in an unconstrained resting state, is shaped as a conical spiral that (a) spirals radially inward from the outer end of the spiral distal portion and (b) defines an outer turn,
pushing of the elongate portion in a distal direction, before the outer turn of the spiral engages cardiac tissue at the target cardiac site, brings an apex and a distal portion of the spiral distal portion in contact with the cardiac tissue, and
further pushing of the elongate portion in a distal direction, after the outer turn of the spiral engages cardiac tissue at the target cardiac site, brings the outer turn of the spiral in contact with the tissue, thereby distributing the distally-directed force against the tissue around the outer turn of the spiral.

For some applications, the distal part of the spiral has soft mechanical characteristics, so that when approaching a wall, or a smaller cavity, the distal part first approaches the target tissue and then deforms under pressure, until the base of the spiral touches the tissue.

For some applications, the cardiac guidewire is configured such that when the spiral distal portion is in the unconstrained resting state, a distal end portion of the elongate portion falls in, is parallel to, or defines an angle of less than 90 degrees with a best-fit plane defined by the outer turn of the spiral.

For some applications, the cardiac guidewire is configured such that when the spiral distal portion is in the unconstrained resting state, a distal end portion of the elongate portion defines an angle of 75-90 degrees with respect to a best-fit plane defined by the outer turn of the spiral.

For some applications, the cardiac guidewire is configured such that when the spiral distal portion is in the unconstrained resting state, the conical spiral has a height of at least 3 mm.

For some applications, the cardiac guidewire is configured such that when the spiral distal portion is in the unconstrained resting state, the conical spiral has a height that equals at least 10% of a diameter of the outer turn of the spiral.

Reference is still made to FIGS. 13C, 15A-B, 17A-C, 18A-B, and 19A-C. In some applications of the present invention, a method is provided that comprises:
intravascularly advancing a cardiac guidewire toward a target cardiac site, the cardiac guidewire shaped so as to define (a) an elongate portion, which is straight when unconstrained, and (b) a spiral distal portion, which, at an outer end thereof, extends from and is supported by a distal end of the elongate portion, wherein the cardiac guidewire is configured such that the spiral distal portion, when in an unconstrained resting state, is shaped as a conical spiral that (i) spirals radially inward from the outer end of the spiral distal portion and (ii) defines an outer turn:
pushing the elongate portion in a distal direction, before the outer turn of the spiral engages cardiac tissue at the target cardiac site, so as to bring an apex and a distal portion of the spiral distal portion in contact with the cardiac tissue: and.
further pushing the elongate portion in a distal direction, after the outer turn of the spiral engages cardiac tissue at the target cardiac site, so as to bring the outer turn of the spiral in contact with the tissue, thereby distributing the distally-directed force against the tissue around the outer turn of the spiral.

For some applications, the cardiac tissue is cardiac tissue of a left atrial appendage (LAA), cardiac tissue of a left ventricle, or cardiac tissue of an ascending aorta.

For some applications, the cardiac tissue is cardiac tissue is of a left ventricle for a TMVR (Transcatheter Mitral Valve repair and replacement) procedure, cardiac tissue of a left ventricle for a TAVR (Transcatheter Aortic Valve repair and replacement) procedure, or cardiac tissue of a right ventricle for TTVR (Transcatheter Tricuspid Valve repair and replacement).

In an application of the present invention, guidewire 20 is connected to a generator comprising sensing and pacing capabilities, and guidewire 20 is used for rapid pacing, temporary pacing, semi-permanent pacing, and/or long-term pacing.

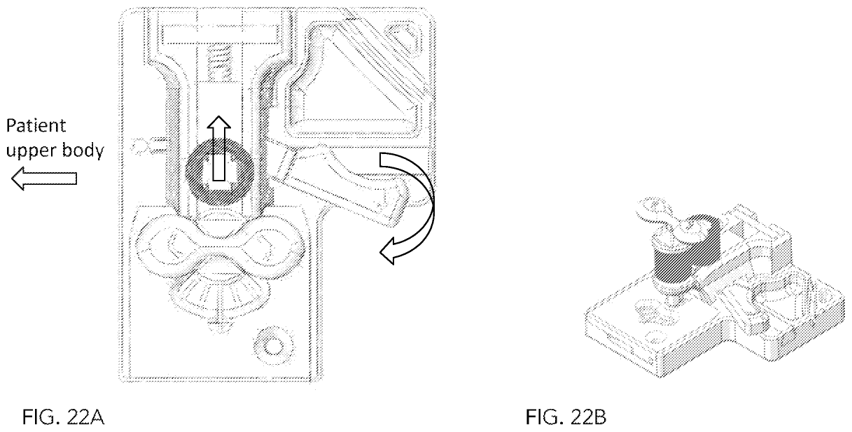

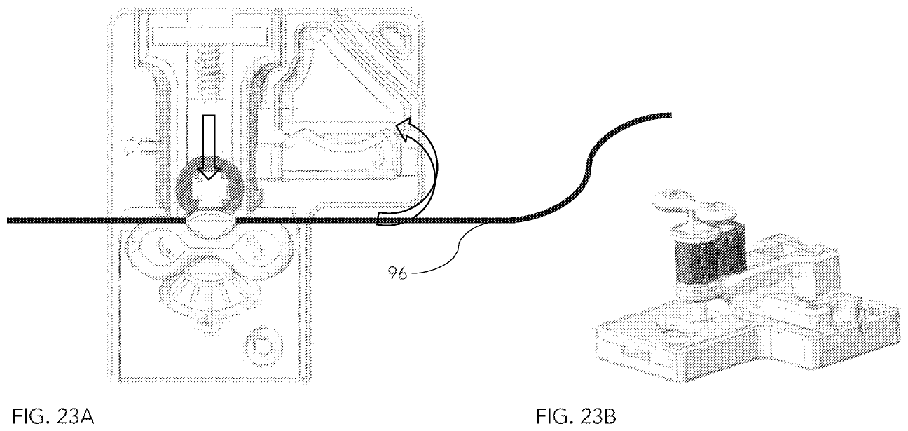

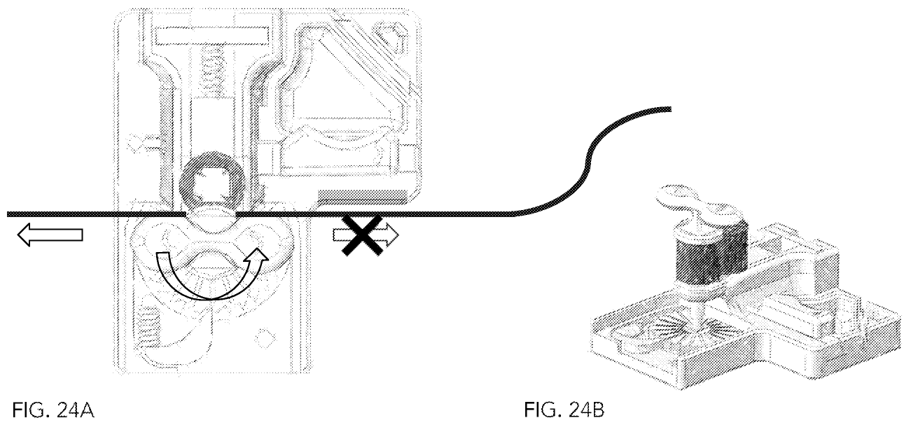

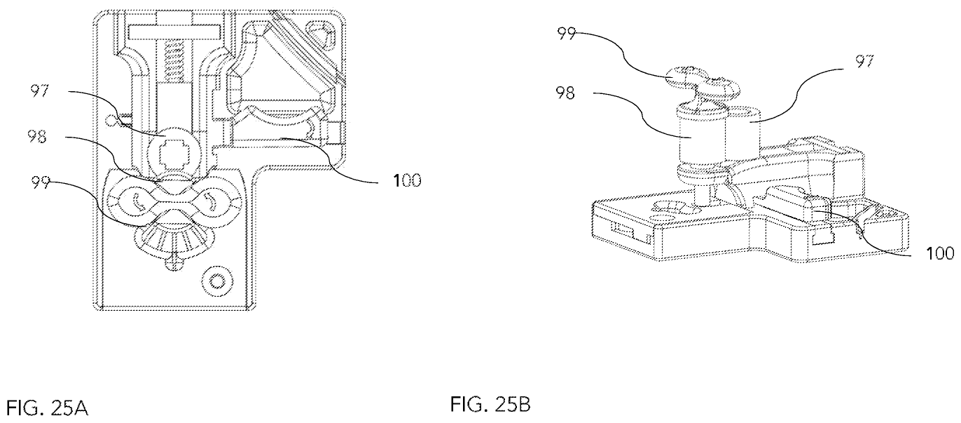

Reference is now made to FIGS. 22A-25B, which are schematic illustrations of a guidewire holder, in accordance with an application of the present invention. The guidewire holder provides a supporting structure for an elongate element, such as a guidewire 96 (labeled in FIG. 23A), catheter, shaft, or a tubular medical instrument, to be spatially stabilized by friction by soft elements 97 and 98, with friction element 97 being pressed against element 98 by a spring mechanism, and guidewire 96 being blocked between elements 97 and 98. A first knob 99, connected to element 98, allows manual rotation of element 98, causing relative advancement of the stabilized guidewire 96 by the applied friction. A second knob 100 is manually movable to allow compression of element 97 against the spring mechanism, thereby allowing creation of a space between elements 97 and 98 during the insertion of guidewire 96, and then, when manually released, allows the compression of element 97 against element 98 and the stabilization of guidewire 96 by friction between elements 97 and 98.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A cardiac guidewire, which is advanceable to a target cardiac site, and which is shaped so as to define:
an elongate portion, which is straight when unconstrained, and
a spiral distal portion, which extends from and is supported by a distal end of the elongate portion,
wherein the cardiac guidewire is configured such that:
the spiral distal portion, when in an unconstrained resting state, is shaped as a spiral that (a) spirals radially outward at an increasing distance from the distal end of the elongate portion so as to define an outer turn, and (b) has a zero or non-zero height, and
pushing of the elongate portion in a distal direction, when the outer turn of the spiral engages cardiac tissue at the target cardiac site, changes the height of the spiral and deforms the spiral.

2. The cardiac guidewire according to claim 1, wherein the cardiac guidewire is configured such that:
the spiral distal portion, when in the unconstrained resting state, is shaped as a planar spiral having the zero height,
the pushing of the elongate portion in the distal direction, when the outer turn of the spiral engages the cardiac tissue, increases the height of the spiral and deforms the spiral into a conical spiral having an apex that faces distally, and
pulling of the elongate portion in a proximal direction, when the outer turn of the spiral engages the cardiac tissue, increases the height of the spiral and deforms the spiral into a conical spiral in which the apex faces proximally.

3. The cardiac guidewire according to claim 1, wherein an interface portion of the cardiac guidewire between the elongate portion and the spiral distal portion has a radius of curvature of 5-30 mm when the spiral distal portion is in the unconstrained resting state.

4. The cardiac guidewire according to claim 1, wherein the spiral distal portion, when in the unconstrained resting state, has 1-6 turns.

5. The cardiac guidewire according to claim 1, wherein a pitch of the spiral distal portion, when in the unconstrained resting state, varies along a central longitudinal axis of the spiral distal portion.

6. The cardiac guidewire according to claim 1, wherein the elongate portion has a length of at least 70 cm.

7. The cardiac guidewire according to claim 1, wherein a distal end of the spiral distal portion, when in the unconstrained resting state, is shaped so as to define a hook.

8. The cardiac guidewire according to claim 1, wherein the cardiac guidewire is configured such that the spiral distal portion, when in the unconstrained resting state, is shaped as a conical spiral having the non-zero height.

9. The cardiac guidewire according to claim 8, wherein the cardiac guidewire is configured such that when the spiral distal portion is in the unconstrained resting state, the non-zero height of the spiral is at least 3 mm.

10. The cardiac guidewire according to claim 8, wherein the cardiac guidewire is configured such that when the spiral distal portion is in the unconstrained resting state, the non-zero height of the spiral equals at least 10% of a diameter of the outer turn of the spiral.

11. The cardiac guidewire according to claim 8, wherein the cardiac guidewire is configured such that when the spiral distal portion is in the unconstrained resting state, the spiral distal portion is shaped as a conical spiral having an apex that faces proximally, such that the pushing of the elongate portion in the distal direction, when the outer turn of the spiral engages the cardiac tissue at the target cardiac site, decreases the height of the conical spiral or inverts the conical spiral such that the apex faces distally.

12. The cardiac guidewire according to claim 11, wherein the cardiac guidewire is configured such that when the spiral distal portion is in the unconstrained resting state, a distal end portion of the elongate portion defines an angle of 75-90 degrees with respect to a best-fit plane defined by the outer turn of the spiral.

13. The cardiac guidewire according to claim 8, wherein the cardiac guidewire is configured such that when the spiral distal portion is in the unconstrained resting state, the spiral distal portion is shaped as a conical spiral having an apex that faces distally, such that the pushing of the elongate portion in the distal direction, when the outer turn of the spiral engages the cardiac tissue at the target cardiac site, increases the height of the conical spiral.

14. The cardiac guidewire according to claim 13, wherein the cardiac guidewire is configured such that when the spiral distal portion is in the unconstrained resting state, a distal end portion of the elongate portion defines an angle of 75-90 degrees with respect to a best-fit plane defined by the outer turn of the spiral.

15. The cardiac guidewire according to claim 8, wherein the conical spiral is a hyperbolic conical spiral, which tapers hyperbolically from a base of the conical spiral to an apex of the conical spiral.

16. The cardiac guidewire according to claim 1, wherein the cardiac guidewire is configured such that when the spiral distal portion is in the unconstrained resting state, a distal end portion of the elongate portion defines an angle of 75-90 degrees with respect to a best-fit plane defined by the outer turn of the spiral.

17. The cardiac guidewire according to claim 16, wherein the angle is 85-90 degrees.

18. The cardiac guidewire according to claim 1, wherein the elongate portion and the spiral distal portion have different stiffnesses.

19. The cardiac guidewire according to claim 18, wherein the elongate portion is stiffer than the spiral distal portion.

20. The cardiac guidewire according to claim 18, wherein the spiral distal portion is stiffer than the elongate portion.

21. The cardiac guidewire according to claim 1, wherein a radially-inner portion of the spiral distal portion and a radially-outer portion of the spiral distal portion have different stiffnesses.

22. The cardiac guidewire according to claim 21, wherein the radially-inner portion of the spiral distal portion is stiffer than the radially-outer portion of the spiral distal portion.

23. The cardiac guidewire according to claim 22, wherein a wire diameter of the spiral distal portion narrows toward a distal end of the spiral distal portion.

24. The cardiac guidewire according to claim 21, wherein the radially-outer portion of the spiral distal portion is stiffer than the radially-inner portion of the spiral distal portion.

25. The cardiac guidewire according to claim 24, wherein a wire diameter of the spiral distal portion widens toward a distal end of the spiral distal portion.

26. A guidewire system comprising the cardiac guidewire according to claim 1, the cardiac guidewire system further comprising a delivery sheath in which the cardiac guidewire is removably disposed in a constrained delivery configuration, with the spiral distal portion held in an unwound elongate configuration by the delivery sheath.

27-82. (canceled)
